(19) United States
(12) Patent Application Publication
(10) Pub. No.: US 2017/0022215 A1
(43) Pub. Date: Jan. 26, 2017

(54) Compounds for Eradicating or Inhibiting Proliferation of Cancer Stem Cells

(71) Applicant: Godavari Biorefineries Limited, Mumbai (IN)

(72) Inventors: Sangeeta Srivastava, Mumbai (IN); Maithili Athavale, Mumbai (IN); Kedar Shukre, Mumbai (IN); Gayatri More, Mumbai (IN)

(21) Appl. No.: 15/124,588

(22) PCT Filed: Mar. 11, 2015
(86) PCT No.: PCT/IN2015/050019

§ 371 Date: Sep. 8, 2016

(30) Foreign Application Priority Data
Mar. 11, 2014 (IN) 814/MUM/2014

(51) Int. Cl. C07D 495/04

(52) U.S. Cl. CPC C07D 495/04

(57) ABSTRACT

The present invention provides compounds of formula (I), compositions, uses thereof and methods for eradicating or inhibiting proliferation of cancer stem cells which includes killing; and/or inducing apoptosis in cancer stem cells. Included within the scope of such compounds, compositions, uses thereof and methods are those in which proliferation of cancer stem cells are selectively eradicated or inhibited.

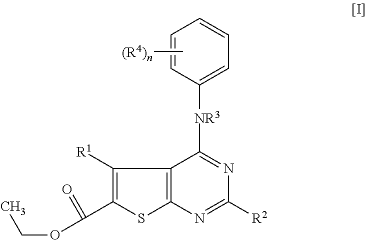

### FIELD OF THE INVENTION

The present invention relates to compounds for eradicating or inhibiting proliferation of cancer stem cells and uses thereof in eradicating or inhibiting proliferation of cancer stem cells. The present invention also relates to a method of eradicating or inhibiting proliferation of cancer stem cells.

### BACKGROUND OF THE INVENTION

Cancer is considered to be the most dreadful disease until today and recurrence or relapse of cancer still remains challenging with the most of the conventional cancer therapies. Radiotherapy is believed to reduce the rate of recurrence to some extent, but it also damages the normal rapidly dividing cells in the area being treated and has never been found to increase overall survival but rather increase mortality. It is also known that though many types of cancer can initially be targeted with chemotherapy using currently available drugs. However, often resistance to treatment with such a drug can occur and recurrence or relapse of cancer is common.

In recent years, a new model for genesis of cancer has gained wide acceptance, it is hypothesized that only a small fraction of cells of the entire tumor mass are responsible for the tumorigenic activities within the tumor. This small fraction of tumorigenic cells, according to the new model, are transformed cells with stem-cell-like qualities and are called “cancer stem cells” (CSCs). In 1990s in vivo presence of CSCs in acute myeloid leukemia (AML) was demonstrated. Later, these CSCs were shown to have the same cellular markers, CD34⁺/CD38″, as that of hematopoietic stem cells. Since then, researchers have conclusively found cancer stem cells in various types of tumors including those of the brain, breast, kidney, skin, prostate, and others.

Studies have demonstrated cancer stem cells to be fundamentally responsible for genesis of cancer, cancer metastasis, and cancer reoccurrence. Cancer stem cells in fact, appear to be resistant to radiotherapy and also refractory to chemotherapeutic and targeted drugs. Normal somatic stem cells appear to be resistant to chemotherapeutic agents as they have various pumps (such as MDR) that pump out drugs, DNA repair proteins and have a slow rate of cell turnover while chemotherapeutic agents target rapidly replicating cells. Cancer stem cells are also believed to have similar mechanisms that allow them to survive drug therapies and radiation treatment, as cancer stem cells are considered to be the mutated counterparts of normal stem cells. It has been postulated that conventional chemotherapies and radiotherapies kill differentiated or differentiating cells, while the population of cancer stem cells that give rise to the differentiated and differentiating cells, could survive and cause a relapse of the disease. Further, it may be likely that chemotherapeutic treatment leaves only chemotherapy-resistant cancer stem cells, and the ensuing recurrent tumor would also be resistant to chemotherapy.

Hence, there is an unmet need of a cancer therapy that can selectively target cancer stem cells to minimize or prevent recurrence or relapse of refractory cancers and tumor metastasis. The same can help to improve survival and the quality of life of cancer patients.

### SUMMARY

The present invention provides compounds having of formula I. In one embodiment, the present invention provides compounds of formula I or a pharmaceutically acceptable derivatives thereof for eradicating or inhibiting proliferation of cancer stem cells, wherein:

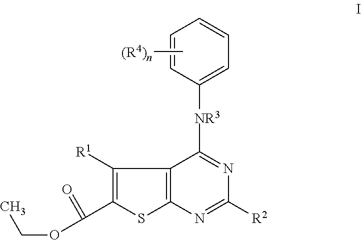

each R¹, R² and R³ is independently selected from halogen, C1-6haloalkyl, —CN, —NO₂, —R, —OR, —SR, —N(R)₂, —N(R)NR₂, —C(NR)NR₂, —N(R)C(O)R, C(O)RN(R)₂, —N(R)C(O)N(R)₂, —N(R)C(O)OR, —OC(O)N(R), —N(R)SO₂R, —SO₂RN(R)₂, C(O)R, —C(O)OR, —OC(O)R, —C(O)OR, —S(O)R, or —SO₂R;
each R is independently selected from H, or an optionally substituted group selected from C1-6 aliphatic, a 3-12 membered saturated or partially unsaturated monocyclic carbocyclic ring, phenyl, an 8-12 membered bicyclic aromatic carbocyclic ring; a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; and
R⁴ is independently selected from —R, —CN, halogen, C1-6haloalkyl, —NO₂, —SR, —N(R)₂, —N(R)NR₂, —C(NR)NR₂, —N(R)C(O)R, C(O)RN(R)₂, —N(R)C(O)N(R)₂, —N(R)C(O)OR, —OC(O)N(R), —N(R)SO₂R, —SO₂RN(R)₂, C(O)R, —C(O)OR, —C(O)OR, —S(O)R, or —SO₂R;
each R is independently selected from H, or an optionally substituted group selected from C1-6 aliphatic, a 3-12 membered saturated or partially unsaturated monocyclic carbocyclic ring, phenyl, an 8-12 membered bicyclic aromatic carbocyclic ring; a 4-8 membered saturated or partially unsaturated monocyclic heterocyclic ring having 1-2 heteroatoms independently selected from nitrogen, or sulfur, a 5-6 membered monocyclic heteroaromatic ring having 1-4 heteroatoms independently selected from nitrogen or sulfur, or an 8-10 membered bicyclic heteroaromatic ring having 1-5 heteroatoms independently selected from nitrogen or sulfur; and

Each n is independently 0-5. In certain embodiments, n is 1-4. In some embodiments, n is 1-3. In yet other embodiments n is 1-2. In some embodiments, n is 0, 1, 2, 3, 4 or 5.

Compounds of the present invention include those described generally above, and are further illustrated by the classes, subclasses, and species disclosed herein. Various terms and terminology used hereinabove in describing the compounds of the present invention and all technical and scientific terms used herein have the same or would mean or refer to standard definition or meaning or as used in a chemical or technical field or as known or commonly understood by one of ordinary skill in the art to which this invention belongs.

Compounds of the present invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄C(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄S(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulphur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{}, -(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)₀₋₂NH₂, —(CH₂)₀₋₂NHR^{}, —(CH₂)₀₋₂NR^{}₂, —NO₂, —SiR₃, —OSiR^{}₃, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR, —O(C(R*₂))₂₋₃O—, or —S(C(R*₂))₂₋₃S—, wherein each independent occurrence of R^{∘} is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

In one embodiment, the compound provided herein is a pharmaceutically acceptable salt of the compound of formula (I). In one embodiment, the compound provided herein is a solvate of the compound of formula (I). In one embodiment, the compound provided herein is a hydrate of compound of formula (I).

In one embodiment, the present invention provides a pharmaceutically acceptable derivative of compound of the formula II or III for eradicating or inhibiting proliferation of cancer stem cells:

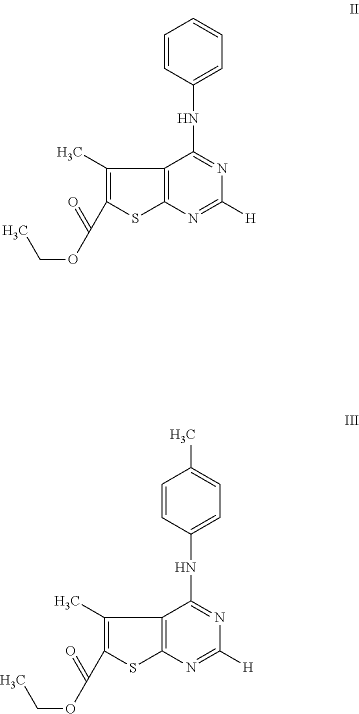 [SMILES: CCOC(=O)c1sc2ncnc(Nc3ccccc3)c2c1C ; CCOC(=O)c1sc2ncnc(Nc3ccc(C)cc3)c2c1C]

In one embodiment, the compound provided herein is a pharmaceutically acceptable salt, ester, a salt of an ester of compound of formula (II) or (III).

According to another aspect, the invention provides a composition comprising a compound of formula (I) or (II) or (III) or a pharmaceutically acceptable salt or derivative thereof and a pharmaceutically acceptable excipient including carrier, adjuvant, or vehicle.

In certain embodiments, a composition comprises a compound having the general formula I, or a pharmaceutically acceptable salt or derivative thereof and a pharmaceutically acceptable excipient, carrier, adjuvant, or vehicle.

In certain embodiments, a composition comprises a compound of the formula (II) or a pharmaceutically acceptable salt or derivative thereof and a pharmaceutically acceptable excipient carrier, adjuvant, or vehicle.

In certain embodiments, a composition comprises a compound of the formula (III) or a pharmaceutically acceptable salt or derivative thereof and a pharmaceutically acceptable excipient carrier, adjuvant, or vehicle.

Such compositions deliver amounts effective for eradicating or inhibiting proliferation of cancer stem cells in a biological sample or in a subject in the need thereof. In certain embodiments, the amount of compound in compositions of this invention is such that it is effective to eradicate or inhibit proliferation of cancer stem cells, in a biological sample or in a subject in the need thereof.

In some embodiments the compound of formula (I) or (II) or (III) or a pharmaceutically acceptable salt or derivative thereof or a composition comprising the said compound is used in eradicating or inhibiting proliferation of cancer stem cells.

In certain embodiments, the invention provides a method of eradicating or inhibiting proliferation of cancer stem cells in a patient, comprising administering to said patient a compound of formula (I) or (II) or (III) or derivative thereof or a composition comprising the said compound in a therapeutically effective amount.

In certain embodiments, the invention provides a method of eradicating or inhibiting proliferation of cancer stem cells leading to remission of the cancer by administering a compound of formula (I) or (II) or (III) or derivative thereof or a composition comprising the same in a therapeutically effective amount to a subject in the need thereof.

In one more embodiments the present invention provides a method of treating disorders or diseases or conditions associated with proliferation of cancer stem cells by administering a compound of formula (I) or (II) or (III) or derivative thereof or a composition comprising the same in a therapeutically effective amount to a subject in the need thereof. Such disorders or diseases include without limitation: cancers, including said cancer occurring in the patient's prostate, breast, skin, muscle, cervical, colon, stomach, liver, pancreas, thyroid, parathyroid, pituitary, thymus, spleen, head, neck, throat, trachea, gall bladder, salivary gland, adrenal gland, esophagus, lymph nodes, sweat glands, sebaceous glands, lung, heart, brain, kidney, ovary, testicle, penis, retina, uvea, conjunctiva, rectum, blood, or bone marrow.

In certain embodiments, the invention provides a method of eradicating or inhibiting proliferation of cancer stem cells in a patient, leading to remission of the cancer, comprising the step of administering to said patient a compound of formula (I) or (II) or (III) or derivative thereof or a composition comprising the same in a therapeutically effective amount to a subject in the need thereof.

In one embodiment, the invention provides a method of eradicating or inhibiting proliferation of cancer stem cells for minimizing or preventing relapse of cancer, comprising administering a compound of formula (I) or (II) or (III) or derivative thereof or a composition comprising the same in a therapeutically effective amount to a subject in the need thereof.

The therapeutically effective amounts of the compounds or compositions comprising the therapeutically effective concentrations of the compounds are formulated into a suitable dosage form to be administered orally, parenterally, by inhalation spray, topically, rectally, nasally, buccally, vaginally or via an implanted reservoir to a subject in the needthereof in practicing the methods. The amounts are effective to eradicate or inhibit proliferation of cancer stem cells.

Depending upon the particular condition, or disease, to be treated, additional therapeutic agents, which are normally administered to treat that condition, may be administered in combination with compounds and compositions of this invention. In some embodiments, a compound of formula (I) or (II) or (III) or derivative thereof is administered in combination with one or more other chemotherapeutic agents.

Other examples of agents that may be combined with compounds of this invention include, without limitation: vitamins and nutritional supplements, cancer vaccines, antisense agents, a monoclonal or polyclonal antibody, an siRNA therapeutic or other agents for treatments of conditions, disorders or diseases other than cancer. In one embodiment, such other agent includes one or more anti-proliferative agents, anti-inflammatory agents, immunomodulatory agents or immunosuppressive agents.

These additional agents may be administered separately from the compound of the formula (I) or (II) or (III) or the derivative thereof or a composition comprising the same as part of a multiple dosage regimen. Alternatively, those agents may be part of a single dosage form, mixed together with the compound of the formula (I) or (II) or (III) or the derivative thereof in a single composition. If administered as part of a multiple dosage regime, the two or more active agents may be submitted simultaneously, sequentially or within a specific period of time from one another, normally within five hours from one another. The amount of both, the compound of the formula (I) or (II) or (III) or the derivative thereof and additional therapeutic agent (in those compositions which comprise an additional therapeutic agent as described above) that may be combined with the pharmaceutically acceptable excipient or carrier materials to produce a single dosage form will vary depending upon the host treated and the particular mode of administration.

These and other aspects of the subject matter described herein will become evident upon reference to the following detailed description.

### BRIEF DESCRIPTION OF THE DRAWINGS

FIG. 1A is a FSC-SSC graph of viable MDA MB231 cells without drug treatment, stained with anti-CD44-PE labeled and anti-CD24-FITC labeled antibodies.

FIG. 1B is the quadrant plot showing a good number of cells (98.5%) expressing CD44 indicating a population rich with cells having stemness property that is cancer stem cells.

FIG. 2A is a FSC-SSC graph of MDA MB231 cells treated with IC25 drug conc. of Cisplatin, stained with anti-CD44-PE labeled and anti-CD24-FITC labeled antibodies.

FIG. 2B is the quadrant plot showing that the exposure of Cisplatin IC25 drug conc. did not have much effect on CD44 expressing cell population of MDA MB231 cells indicating that Cisplatin is not very effective on CD44 expressing population that is cancer stem cells.

FIG. 3A is a FSC-SSC graph of MDA MB231 cells treated with IC25 drug conc. of Ethyl-5-methyl-4-(phenylamino) thieno[2,3-d]pyrimidine-6-carboxylate, stained with anti-CD44-PE labeled and anti-CD24-FITC labeled antibodies.

FIG. 3B is the quadrant plot showing that the exposure of Ethyl-5-methyl-4-(phenylamino) thieno[2,3-d]pyrimidine-6-carboxylate IC25 drug conc. had a marked effect on the CD44 expressing population of MDA MB231 cells indicating that Ethyl-5-methyl-4-(phenylamino) thieno[2,3-d]pyrimidine-6-carboxylate is very effective on CD44 population that is cancer stem cells population in breast cancer cells.

FIG. 4A is a FSC-SSC graph of MDA MB231 cells treated with IC25 drug conc. of Ethyl-5-methyl-4-(4-methylphenyl)amino thieno[2,3-d]pyrimidine-6-carboxylate, stained with anti-CD44-PE labeled and anti-CD24-FITC labeled antibodies.

FIG. 4B is the quadrant plot showing that the exposure of Ethyl-5-methyl-4-(4-methylphenyl)amino thieno[2,3-d]pyrimidine-6-carboxylate) IC25 drug conc. had a marked effect on the CD44 expressing population of MDA MB231 cells indicating that Ethyl-5-methyl-4-(4-methylphenyl)amino thieno[2,3-d]pyrimidine-6-carboxylate) is very effective on CD44 expressing population that is cancer stem cells population in breast cancer cells.

FIG. 5 is a bar graph showing that Ethyl-5-methyl-4-(phenylamino) thieno[2,3-d]pyrimidine-6-carboxylate and Ethyl-5-methyl-4-(4-methylphenyl)amino thieno[2,3-d]pyrimidine-6-carboxylate exhibited much better and enhanced activity on CD44 expressing cells that is cancer stem cells population of MDA MB231 cancer cells compared to standard therapeutic drug Cisplatin.

FIG. 6A is a FSC-SSC graph of viable DU145 cells without drug treatment, stained with anti-CD44-PE labeled and anti-CD24-FITC labeled antibodies.

FIG. 6B is the quadrant plot showing a good number of cells (98.5%) expressing CD44 indicating a population rich with cells having stemness property that is cancer stem cells.

FIG. 7A is a FSC-SSC graph of DU145 cells treated with IC25 drug conc. of Cisplatin, stained with anti-CD44-PE labeled and anti-CD24-FITC labeled antibodies.

FIG. 7B is the quadrant plot showing that the exposure of Cisplatin IC25 drug conc. did not have much effect on CD44 expressing cell population of DU145 cells indicating that Cisplatin is not very effective on CD44 expressing population that is cancer stem cells.

FIG. 8A is a FSC-SSC graph of DU145 cells treated with IC25 drug conc. of Ethyl-5-methyl-4-(phenylamino) thieno[2,3-d]pyrimidine-6-carboxylate, stained with anti-CD44-PE labeled and anti-CD24-FITC labeled antibodies.

FIG. 8B is the quadrant plot showing that the exposure of Ethyl-5-methyl-4-(phenylamino) thieno[2,3-d]pyrimidine-6-carboxylate IC25 drug conc. had a marked effect on the CD44 expressing population of DU145 cells indicating that Ethyl-5-methyl-4-(phenylamino) thieno[2,3-d]pyrimidine-6-carboxylate is very effective on CD44 population that is cancer stem cells population in prostate cancer cells.

FIG. 9A is a FSC-SSC graph of DU145 cells treated with IC25 drug conc. of Ethyl-5-methyl-4-(4-methylphenyl)amino thieno[2,3-d]pyrimidine-6-carboxylate, stained with anti-CD44-PE labeled and anti-CD24-FITC labeled antibodies.

FIG. 9B is the quadrant plot showing that the exposure of ethyl-5-methyl-4-(4-methylphenyl)amino thieno[2,3-d]pyrimidine-6-carboxylate) IC25 drug conc. had a marked effect on the CD44 expressing population of DU145 cells indicating that Ethyl-5-methyl-4-(4-methylphenyl)amino thieno[2,3-d]pyrimidine-6-carboxylate) is very effective on CD44 expressing population that is cancer stem cells population in prostate cancer cells.

FIG. 10 is a bar graph showing that Ethyl-5-methyl-4-(phenylamino) thieno[2,3-d]pyrimidine-6-carboxylate and Ethyl-5-methyl-4-(4-methylphenyl)amino thieno[2,3-d]pyrimidine-6-carboxylate exhibits much better and enhanced activity on CD44 expressing cells that is cancer stem cells population of DU145 cancer cells compared to standard therapeutic drug Cisplatin.

### DETAILED DESCRIPTION OF THE INVENTION

All scientific and technical terms used herein unless defined otherwise, have the same meaning as is commonly understood by one of ordinary skill in the art.

In one embodiment, the compounds provided herein are of formula I. In one embodiment, the compounds provided herein are of formula I or derivative thereof. In one embodiment the present invention provides compounds having the general formula I or a pharmaceutically acceptable salts, solvates, or hydrates thereof for eradicating or inhibiting proliferation of cancer stem cells, wherein:

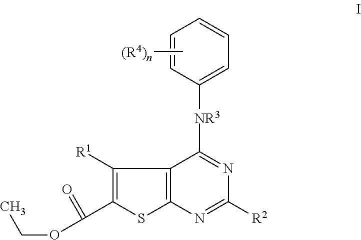

each R¹, R² and R³ is independently selected from halogen, C1-6haloalkyl, —CN, —NO₂, —R, —OR, —SR, —N(R)₂, —N(R)NR₂, —C(NR)NR₂, —N(R)C(O)R, C(O)RN(R)₂, —N(R)C(O)N(R)₂, —N(R)C(O)OR, —OC(O)N(R), —N(R)SO₂R, —SO₂RN(R)₂, C(O)R, —C(O)OR, —OC(O)R, —C(O)OR, —S(O)R, or —SO₂R;
each R is independently selected from H, or an optionally substituted group selected from C1-6 aliphatic, a 3-12 membered saturated or partially unsaturated monocyclic carbocyclic ring, phenyl, an 8-12 membered bicyclic aromatic carbocyclic ring; a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; and
R⁴ is independently selected from —R, —CN, halogen, C1-6haloalkyl, —NO₂, —SR, —N(R)₂, —N(R)NR₂, —C(NR)NR₂, —N(R)C(O)R, C(O)RN(R)₂, —N(R)C(O)N(R)₂, —N(R)C(O)OR, —OC(O)N(R), —N(R)SO₂R, —SO₂RN(R)₂, C(O)R, —C(O)OR, —C(O)OR, —S(O)R, or —SO₂R;
each R is independently selected from H, or an optionally substituted group selected from C1-6 aliphatic, a 3-12 membered saturated or partially unsaturated monocyclic carbocyclic ring, phenyl, an 8-12 membered bicyclic aromatic carbocyclic ring; a 4-8 membered saturated or partially unsaturated monocyclic heterocyclic ring having 1-2 heteroatoms independently selected from nitrogen, or sulfur, a 5-6 membered monocyclic heteroaromatic ring having 1-4 heteroatoms independently selected from nitrogen or sulfur, or an 8-10 membered bicyclic heteroaromatic ring having 1-5 heteroatoms independently selected from nitrogen or sulfur; and

Each n is independently 0-5. In certain embodiments, n is 1-4. In some embodiments, n is 1-3. In yet other embodiments n is 1-2. In some embodiments, n is 0, 1, 2, 3, 4 or 5.

Compounds of the present invention include those described generally above, and are further illustrated by the classes, subclasses, and species disclosed herein. Various terms and terminology used hereinabove in describing the compounds of the present invention and all technical and scientific terms used herein have the same or would mean or refer to standard definition or meaning or as used in a chemical or technical field or as known or commonly understood by one of ordinary skill in the art to which this invention belongs.

Compounds of the present invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄C(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄S(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O-N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulphur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{}, -(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)₀₋₂NH₂, —(CH₂)₀₋₂NHR^{}, —(CH₂)₀₋₂NR^{}₂, —NO₂, —SiR₃, —OSiR^{}₃, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

A “pharmaceutically acceptable derivative” means any non-toxic salt, ester, salt of an ester or other derivative of a compound of this invention that, upon administration to a recipient, is capable of providing, either directly or indirectly, a compound of this invention or an active metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Exemplary pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In certain embodiments, a warhead moiety, R¹, of a provided compound comprises one or more deuterium atoms.

In one embodiment, the compounds of formula I is not compound of formula II or III.

In certain embodiments, the present invention provides a pharmaceutically acceptable derivative of compound of the formula II or III for eradicating or inhibiting proliferation of cancer stem cells:

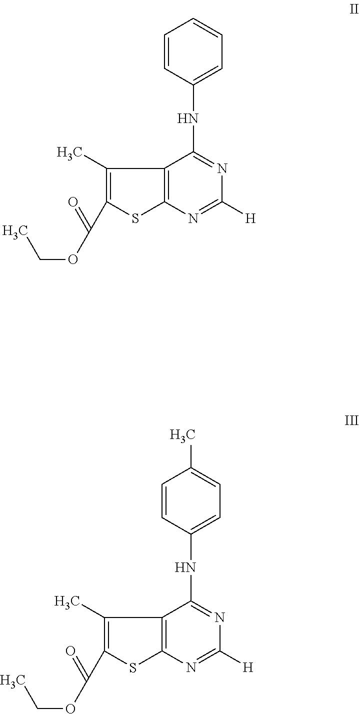 [SMILES: CCOC(=O)c1sc2ncnc(Nc3ccccc3)c2c1C ; CCOC(=O)c1sc2ncnc(Nc3ccc(C)cc3)c2c1C]

In one embodiment, the compound provided herein is a pharmaceutically acceptable salt, ester, a salt of an ester of compound of formula II or III.

In one embodiment, the derivative of compound of formula II is compound of formula IV or formula V:

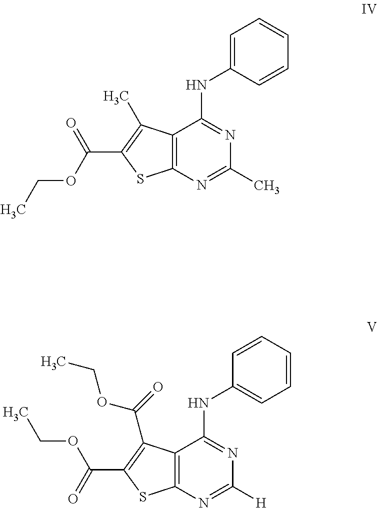 [SMILES: CCOC(=O)c1sc2nc(C)nc(Nc3ccccc3)c2c1C ; CCOC(=O)c1sc2ncnc(Nc3ccccc3)c2c1C(=O)OCC]

In one embodiment the derivative of compound of formula III is compound of formula VI or formula VII:

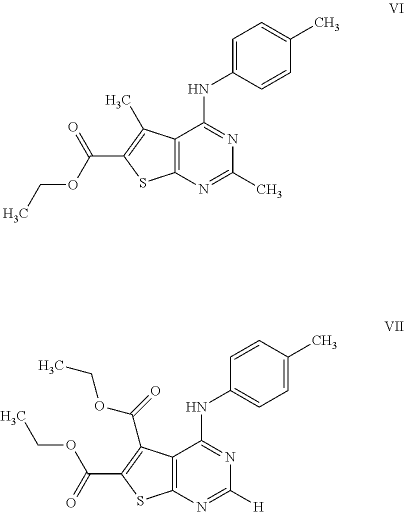

According to another aspect, the invention provides a composition comprising a compound of formula (I) or (II) or (III) or a pharmaceutically acceptable salt or derivative thereof and a pharmaceutically acceptable excipient including carrier, adjuvant, or vehicle.

In certain embodiments, a composition comprises a compound having the general formula I, or a pharmaceutically acceptable salt or derivative thereof and a pharmaceutically acceptable excipient, carrier, adjuvant, or vehicle.

In certain embodiments, a composition comprises a compound of the formula (II) or a pharmaceutically acceptable salt or derivative thereof and a pharmaceutically acceptable excipient carrier, adjuvant, or vehicle.

In certain embodiments, a composition comprises a compound of the formula (III) or a pharmaceutically acceptable salt or derivative thereof and a pharmaceutically acceptable excipient carrier, adjuvant, or vehicle.

The term pharmaceutically acceptable excipient, carrier, adjuvant, or vehicle refers to a non-toxic excipient carrier, adjuvant, or vehicle that does not destroy the pharmacological activity of the compound with which it is formulated. The derivative of the compound of formula (II) or (III) may be a pharmaceutically acceptable ester, or salt of an ester.

The amount of compound in compositions of this invention is such that it is effective for in eradicating or inhibiting proliferation of cancer stem cells, in a biological sample or in a subject in the need thereof. In certain embodiments, the amount of compound in compositions of this invention is such that it is effective to measurably eradicate or inhibit proliferation of cancer stem cells, in a biological sample or in a subject in the need thereof.

A “subject” includes a mammal, preferably a human, but can also be an animal in need of veterinary treatment. The term “subject in the need thereof” refers to a patient suffering from disease, disorder or condition associated with proliferation of cancer stem cells for example any type of cancer or relapse or recurrence of cancer.

In certain embodiments, the composition comprises between the biologically effective dose and the maximum tolerated dose of the compound of formula I, or formula II or formula III or derivatives thereof in a therapeutically effective amount.

In certain embodiments, a composition of this invention can be formulated for administration to a subject in the need thereof. In some embodiments, preferably a composition of this invention can be formulated for oral administration to a patient.

Compositions of the present invention may be formulated into a suitable dosage form to be administered orally, parenterally, by inhalation spray, topically, rectally, nasally, buccally, vaginally or via an implanted reservoir. Compositions of the present invention may be formulated into oral dosage forms including liquid, solid, and semisolid dosage forms. The term “parenteral” as used herein includes subcutaneous, intravenous, intraperitoneal, intramuscular, intra-articular, intra-synovial, intrasternal, intrathecal, intrahepatic, intralesional and intracranial injection or infusion techniques. Preferably, the compositions are administered orally, intravenously or intraperitoneally.

Sterile injectable forms of the compositions of this invention may be sterile injectable aqueous solution or oleaginous suspension in a non-toxic parenterally acceptable diluent or solvent, or suspension, suitable dispersing or wetting agents and suspending agents.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. Depot injectable formulations may also be prepared by entrapping the compound in liposomes or microemulsions that are compatible with body tissues.

Pharmaceutically acceptable compositions of this invention may be orally administered in any orally acceptable dosage form including, but not limited to, capsules, tablets, aqueous suspensions or solutions.

Solid dosage forms for oral administration include but are not limited to capsules, tablets, pills, powders, and granules. In such solid dosage forms, the active compound is mixed with at least one inert, pharmaceutically acceptable excipient or carrier, fillers or extenders, binders, humectants, disintegrating agents, solution retarding agents, absorption accelerators, wetting agents, absorbents, lubricants, buffering agents, and/or mixtures thereof.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and/or emulsifiers. Besides inert diluents, the oral compositions can also include adjuvants such as wetting agents, emulsifying and suspending agents, sweetening, flavoring, and perfuming agents.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

For topical applications, provided pharmaceutically acceptable compositions may be formulated in a suitable ointment containing the active component suspended or dissolved in one or more carriers. Alternatively, provided pharmaceutically acceptable compositions can be formulated in a suitable lotion, gel or cream containing the active components suspended or dissolved in one or more pharmaceutically acceptable carriers.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Additionally, the present invention contemplates the use of transdermal patches, which may have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Ophthalmic formulation, ear drops, and eye drops are also contemplated as being within the scope of this invention. For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative. Alternatively, for ophthalmic uses, the pharmaceutically acceptable compositions may be formulated in an ointment.

Pharmaceutically acceptable compositions of this invention may also be administered by nasal aerosol or inhalation. Such compositions are prepared according to techniques well-known in the art of pharmaceutical formulation and may be prepared as solutions in suitable preservatives, absorption promoters to enhance bioavailability, and/or other conventional solubilizing or dispersing agents.

Topical application for the lower intestinal tract can be effected in a rectal suppository formulation (see above) or in a suitable enema formulation. Topically-transdermal patches may also be used. Compositions for rectal or vaginal administration are preferably suppositories which can be prepared by mixing the compounds of this invention with suitable non-irritating excipients or carriers.

Most preferably, pharmaceutically acceptable compositions of this invention may be formulated for oral administration. Such formulations may be administered with or without food.

The amount of compounds of the present invention that may be combined with the carrier materials to produce a composition in a single dosage form will vary depending upon the subject to be treated, the particular mode of administration. Preferably, provided compositions should be formulated so that an effective dosage of the compound of the invention can be administered to a subject receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

In one embodiment compounds of the present invention having the general formula I or a pharmaceutically acceptable salt thereof or compositions thereof may be used for eradicating or inhibiting proliferation of cancer stem cells and thereby treating associated disorders or diseases or conditions. Thus, provided compounds may be useful for treating cancers, including, but not limited to hematological cancers and solid tumors.

In certain embodiments compounds of the present invention having the formula II or a pharmaceutically acceptable salt thereof or compositions thereof may be used for eradicating or inhibiting proliferation of cancer stem cells and thereby treating associated disorders or diseases or conditions. Thus, provided compounds are useful for treating cancers, including, but not limited to hematological cancers and solid tumors.

In certain embodiments compounds of the present invention having the formula III or a pharmaceutically acceptable salt thereof or compositions thereof may be used for eradicating or inhibiting proliferation of cancer stem cells and thereby treating associated disorders or diseases or conditions. Thus, provided compounds are useful for treating cancers, including, but not limited to hematological cancers and solid tumors.

As used herein, the terms “eradicating or inhibiting proliferation of cancer stem cells” refer to the eradication of cancer stem cells by inhibiting or suppressing growth, division, maturation or viability of cancer stem cells, and/or causing the death of cancer stem cells, individually or in aggregate with other cancer stem cells, by cytotoxicity or the induction of apoptosis. One of skill in the art will appreciate that by definition, “eradicating or inhibiting proliferation of cancer stem cells” also encompasses the eradication or inhibition of the growth, division, maturation or viability of cancer cells, and/or causing the death of cancer cells, individually or in aggregate with other cancer cells, by cytotoxicity or the induction of apoptosis.

In another embodiment the present invention provides a method of eradicating or inhibiting proliferation of cancer stem cells by administering therapeutically effective amount of a compound having the general formula I or a pharmaceutically acceptable salt or derivative thereof or compositions comprising the same in subjects in the need thereof.

In certain embodiments the present invention provides a method of eradicating or inhibiting proliferation of cancer stem cells by administering therapeutically effective amount of the compound having the formula II or a derivative or salt thereof or compositions comprising the same in subjects in the need thereof.

In certain embodiments the present invention provides a method of eradicating or inhibiting proliferation of cancer stem cells by administering therapeutically effective amount of the compound having the formula III or a derivative or a salt thereof or compositions comprising the same in subjects in the need thereof.

The activity of a compound utilized in this invention for eradicating or inhibiting proliferation of cancer stem cells or other cancer cells, may be assayed in vitro or in vivo. An in vivo assessment of the eliminating or cytotoxic activity of the compounds of the invention may be made using an animal model of cancer, e.g., a rodent or primate model. Cell-based assays may be performed using, e.g., a cell line isolated from a tumor or blood-borne cancer. Cell-based assays for activity against a specific protein or nucleic acid component of a cancer cell line. e.g., an enzyme, structural protein, cell surface markers, DNA or RNA, or microarrays, may also be performed. Additionally, biochemical or mechanism-based assays, e.g., transcription assays using a purified protein, Northern blot, RT-PCR, etc., may be performed. In vitro assays include assays that determine cell morphology, viability, cell count, or growth inhibition, and/or the cytotoxicity, enzyme inhibitory activity, and/or the subsequent functional consequences of treatment of cancer cells with compounds of the invention. Alternate in vitro assays quantitate the ability of the compounds of the present invention to bind to protein or nucleic acid molecules within the cell.

Examples of cancer cell lines that may be used for testing the inhibition or may be inhibited by the compounds and compositions described herein and against which the methods described herein may be useful include but are not limited to DU145, LNCaP, PC3, MDA MB 231, MCF7, T47D, L929, HeLa, Bu25tK, Colo320, or other cell lines derived from tissues including, but not limited to, prostate, breast, fibroblast, cervical, colon, liver, pancreas, lung or kidney.

According to one embodiment, the invention relates to a method of eradicating or inhibiting proliferation of cancer stem cells in a biological sample comprising the step of contacting said biological sample with a compound of this invention, or a composition comprising said compound. In certain embodiments, the invention relates to a method of killing cancer stem cells or cancer cells in a biological sample comprising the step of contacting said biological sample with a compound of this invention, or a composition comprising said compound.

The term “compound of this invention” or “compound of the invention”, as used herein, includes the compounds having the formula I, it's derivative or salt or derivative of compound of formula II or formula III.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Eradicating cancer stem cells in a biological sample may be useful for a variety of purposes that are known to one of skill in the art. Examples of such purposes include, but are not limited to biological assays, gene expression studies, and biological target identification.

In one more embodiment the present invention provides a method of treatment of disorders or diseases or conditions associated with cancer stem cells by administering compounds of this invention or compositions comprising the same in an effective amount in subjects in the need thereof.

In certain embodiments, the present invention provides a method for treating a disorder mediated by cancer stem cells, in a patient in need thereof, comprising the step of administering to said patient a compounds of this invention or a composition comprising the same in an effective amount. Such disorders include cancer or recurrence or relapse of cancer.

In certain embodiments, the invention provides a method of eradicating or inhibiting proliferation of cancer stem cells in a patient leading to remission of the cancer, comprising the step of administering to said patient a compound of this invention or a composition comprising said compound in an effective amount.

In one embodiment, the invention provides a method of eradicating or inhibiting proliferation of cancer stem cells for minimizing or preventing relapse or recurrence of cancer, comprising administering compound of this invention or composition comprising the same in an effective amount to a subject in the needthereof.

Cancer includes cancer occurring in the patient's prostate, breast, neck, skin, muscle, colon, liver, stomach, pancreas, kidney, ovary, lung, testicle, penis, thyroid, parathyroid, pituitary, thymus, retina, uvea, conjunctiva, spleen, head, trachea, gall bladder, rectum, salivary gland, adrenal gland, throat, esophagus, lymph nodes, sweat glands, sebaceous glands, heart, brain, blood or bone marrow.

In some embodiments the compounds and compositions of the present invention may be used in a method of treating a cancer or other proliferative disorder. In some embodiments the present invention provides a method of treating a cancer or other proliferative disorder, comprising administering a compound or composition of the present invention to a patient with a cancer or other proliferative disorder. In certain embodiments the compounds and compositions of the present invention may be used to treat a cancer in a mammal. In certain embodiments the mammal is a human patient. In certain embodiments the compounds and compositions of the present invention may be used to treat a cancer in a human patient, said cancer occurring in the patient's prostate, breast, neck, skin, muscle, colon, liver, stomach, pancreas, kidney, ovary, lung, testicle, penis, thyroid, parathyroid, pituitary, thymus, retina, uvea, conjunctiva, spleen, head, trachea, gall bladder, rectum, salivary gland, adrenal gland, throat, esophagus, lymph nodes, sweat glands, sebaceous glands, heart, brain, blood or bone marrow.

In certain embodiments, the invention provides a method of eradicating or inhibiting proliferation of cancer stem cells in a patient leading to treatment, remission or minimizing or preventing recurrence or relapse of the breast cancer, comprising the step of administering to said patient a compound of this invention or a composition comprising said compound in an effective amount.

In certain embodiments, the invention provides a method of eradicating or inhibiting proliferation of cancer stem cells in a patient leading to treatment, remission or minimizing or preventing recurrence or relapse of the prostate cancer, comprising the step of administering to said patient a compound of this invention or a composition comprising said compound in an effective amount.

Depending upon the particular condition, or disease, to be treated, additional therapeutic agents, which are normally administered to treat that condition, may be administered in combination with compounds and compositions of this invention. In some embodiments, a provided compound of this invention, or composition thereof, is administered in combination with one or more other chemotherapeutic agents. Such chemotherapeutic agents include, but are not limited to agents such as kinase inhibitors, alkylating agents, anti-metabolites, tubulin stabilizers, tubulin assembly inhibitors, DNA replication inhibitors, cell cycle inhibitors, topoisomerase inhibitors, cytotoxic antibiotics or nanoparticle or protein conjugates of any of the aforementioned agents.

In certain embodiments, a combination of 2 or more chemotherapeutic agents may be administered together with compounds of the invention. In certain embodiments, a combination of 3 or more chemotherapeutic agents may be administered with compounds of the invention. In some embodiments, the chemotherapeutic agents are selected from alkylating agents or anti-metabolites.

Other examples of agents that may be combined with compounds of this invention include, without limitation: vitamins and nutritional supplements, cancer vaccines, antisense agents, a monoclonal or polyclonal antibody, an siRNA therapeutic or other agents for treatments of conditions, disorders or diseases other than cancer.

In one embodiment, such other agent includes one or more anti-proliferative agents, anti-inflammatory agents, immunomodulatory agents or immunosuppressive agents.

Those additional agents may be administered separately from the compound of the invention-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another, normally within five hours from one another. The amount of both, the compound of this invention and additional therapeutic agent (in those compositions which comprise an additional therapeutic agent as described above) that may be combined with the carrier materials to produce a single dosage form will vary depending upon the host treated and the particular mode of administration.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent.

The amount of additional therapeutic agent present in the compositions of this invention may be no more than the amount that would normally be administered in a composition comprising that therapeutic agent as the only active agent. Preferably the amount of additional therapeutic agent in the presently disclosed compositions may range from about 5% to 90% of the amount normally present in a composition comprising that agent as the only therapeutically active agent.

Resistance to chemotherapeutic drugs is a major factor limiting the efficacy of therapies against many cancers and other proliferative disorders. The rapid division rate of these cells allows for the development of mutations or upregulation of pumps such as MDR that afford resistance to current first line chemotherapy drugs. The problem of relapse of cancers in a more drug-resistant form is a critical hurdle faced in drug development of new chemotherapeutic drugs to treat cancer patients.

The present invention can address this problem by providing the compounds of this invention and compositions thereof for eradicating or inhibiting proliferation of cancer stem cells and thereby treating associated disorders or diseases or conditions in particular for avoiding or minimizing problem of relapse of cancers.

The compounds of the invention may be prepared according to the methods of synthesis that may be known to one of ordinary skilled in the art or can be specifically designed to synthesize compounds of the invention or their subclasses or species of each of these compounds, as described herein.

The foregoing description of the invention has been set merely to illustrate the invention and is not intended to be limiting. Since modifications of the disclosed embodiments including without limitation those relating to the chemical structures, substituents, derivatives, intermediates, syntheses, compositions, formulations and/or methods of use provided herein incorporating the spirit and substance of the invention may occur to person skilled in the art, the invention should be construed to include everything within the scope of the disclosure.

### EXAMPLES

### Example 1

### Preparation of Ethyl-5-methyl-4-(phenylamino) thieno[2,3-d]pyrimidine-6-carboxylate

Three-neck round-bottom flask was arranged with water condenser, thermometer pocket on magnetic stirrer and charged ethylacetoacetate (4 ml), malononitrile (2.48 gm), sulfur (1.2 gm) in methanol (37.5 ml) and morpholine (6.97 ml) under stirring at room temperature. The mixture was stirred at room temperature for 100 mins and then refluxed for 3 hours. The reaction was monitored on TLC, after complete conversion reaction mass allowed to cool at room temperature and filtered under vacuum and the product was washed with methanol to obtained ethyl 5-amino-4-cyano-3-methylthiophene-2-carboxylate.

Similar reaction set up as above was arranged and charged ethyl 5-amino-4-cyano-3-methylthiophene-2-carboxylate (0.210 gm, 1 mmol), 10 ml mixture of formic acid:Conc. hydrochloric acid (1:1) was added and refluxed on water bath for 2 hrs. The reaction was monitored on TLC after completion of reaction, allowed to cool at room temperature and poured onto crushed ice. Obtained solid was filtered under vacuum and washed with water to get pure ethyl 5-methyl-4-oxo-3,4-dihydrothieno[2,3-d]pyrimidine-6-carboxylate.

Similar reaction set up as above was arranged and charged ethyl 5-methyl-4-oxo-3,4-dihydrothieno[2,3-d]pyrimidine-6-carboxylate (0.238 gm 1 mmol), POCL₃ (10 ml) and 2 drops of DMF under stirring. When addition was complete, reaction was refluxed for 1 hr, cooled and poured onto crushed ice. Ethyl 4-chloro-5-methyl thieno [2,3-d]pyrimidine-6-carboxylate was got precipitated out, which was filtered under vacuum and washed it with water and dried to give pure ethyl 4-chloro-5-methyl thieno [2,3-d]pyrimidine-6-carboxylate.

Thus obtained pure ethyl 4-chloro-5-methyl thieno[2,3-d]pyrimidine-6-carboxylate (0.256 gm, 1 mmol) was taken in 50 ml round-bottom flask containing 10 ml ethanol. Flask was arranged on magnetic stirrer was equipped with water condenser, thermometer pocket and charged slowly aniline (0.1 ml 1 mmol) and refluxed for 4 hrs. After completion of reaction, the reaction mixture was allowed to cool at room temperature and poured onto crushed ice, product Ethyl-5-methyl-4-(phenyl amino) thieno [2,3-d]pyrimidine-6-carboxylate got precipitated out which was separated by filtration under vacuum and washed with water and dried to give pure Ethyl-5-methyl-4-(phenyl amino) thieno [2,3-d]pyrimidine-6-carboxylate.

### Example 2

### Preparation of Ethyl-5-methyl-4-(4-methylphenyl)amino thieno[2,3-d]pyrimidine-6-carboxylate

Three-neck round-bottom flask was arranged with water condenser, thermometer pocket on magnetic stirrer and charged ethylacetoacetate (4 ml), malononitrile (2.48 gm), sulfur (1.2 gm) in methanol (37.5 ml) and morpholine (6.97 ml) under stirring at room temperature. The mixture was stirred at room temperature for 10 min. and then refluxed for 3 hours. The reaction was monitored on TLC, after complete conversion reaction mass was allowed to cool at room temperature, filtered under vacuum and washed product with methanol to get ethyl 5-amino-4-cyano-3-methylthiophene-2-carboxylate.

Similar set up as above was arranged and charged ethyl 5-amino-4-cyano-3-methylthiophene-2-carboxylate (0.210 gm, 1 mmol), was added 10 ml mixture of formic acid:Conc. hydrochloric acid (1:1) and refluxed on water bath for 2 hrs. The reaction was monitored on TLC, after completion of reaction the reaction mixture was allowed to cool at room temperature and poured onto crushed ice. Obtained solid was filter under vacuum and wash with water to get pure ethyl 5-methyl-4-oxo-3,4-dihydrothieno[2,3-d]pyrimidine-6-carboxylate.

Set up as mentioned above was arranged and charged ethyl 5-methyl-4-oxo-3,4-dihydrothieno[2,3-d]pyrimidine-6-carboxylate (0.238 gm 1 mmol), POCL₃ 10 ml and 2 drops of DMF under stirring. When addition was complete, reaction was refluxed for 1 hr, cooled and poured onto crushed ice. Ethyl 4-chloro-5-methyl thieno[2,3-d]pyrimidine-6-carboxylate got precipitated out, it was filtered under vacuum and wash it with water and dried to get pure ethyl 4-chloro-5-methyl thieno[2,3-d]pyrimidine-6-carboxylate.

Thus obtained pure ethyl 4-chloro-5-methyl thieno [2,3-d]pyrimidine-6-carboxylate (0.256 gm, 1 mmol) was taken in 50 ml round-bottom flask containing 10 ml ethanol. Flask was arranged on magnetic stirrer and equipped with water condenser, thermometer pocket and charged slowly P-toluidine (0.107 gm, 1 mmol) and refluxed for 4 hrs. After completion of reaction, the reaction mixture was allowed to cool at room temperature and poured onto crushed ice, product Ethyl-5-methyl-4-[(4-methylphenyl)amino]thieno[2,3-d]pyrimidine-6-carboxylate got precipitated out which was separated by filtration under vacuum which, washed with water and dried to give Ethyl-5-methyl-4-[(4-methylphenyl)amino]thieno[2,3-d]pyrimidine-6-carboxylate.

### Example 3

### Flow Cytometry Analysis

### Examples 3A

### Study of Effect of Ethyl-5-methyl-4-(phenylamino) thieno[2,3-d]pyrimidine-6-carboxylate; Ethyl-5-methyl-4-(4-methylphenyl)amino thieno[2,3-d]pyrimidine-6-carboxylate and Cisplatin on Breast Cancer Cell Line MDA MB231 by Flow Cytometry Analysis

MDA MB231 is a highly metastatic breast cancer cell line. Flow cytometry study was conducted to observe the effect of Ethyl-5-methyl-4-(phenylamino) thieno[2,3-d]pyrimidine-6-carboxylate and Ethyl-5-methyl-4-(4-methylphenyl)amino thieno[2,3-d]pyrimidine-6-carboxylate on MDA MB231 cells compared to standard therapeutic drug Cisplatin.

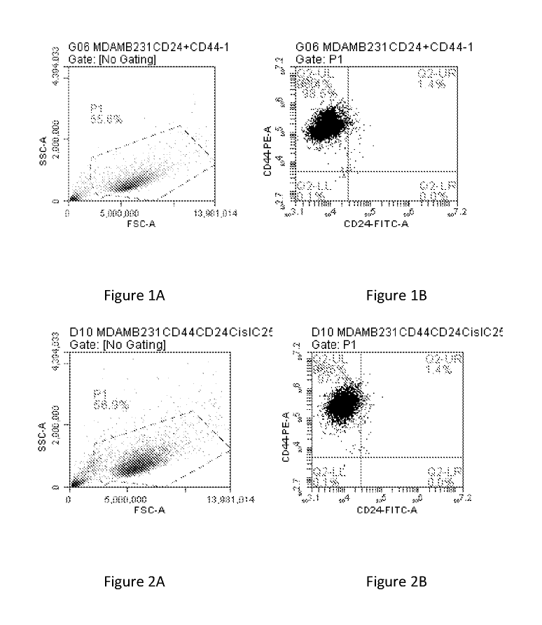

1. Untreated population: MDA MB231 cells without drug treatment were stained with anti-CD44-PE labeled and anti-CD24-FITC labeled antibodies (as seen in FSC-SSC graph FIG. 1A) and the expression was observed in the quadrant plot. In the quadrant plot upper left region (Q2-UL) only CD44 expressing cells were observed. In the quadrant plot upper right region (Q2-UR) CD44 and CD24 expressing cells were observed. In the quadrant plot lower left region (Q2-LL) only CD24 expressing cells were observed. In the quadrant plot lower right region (Q2-LR) neither CD44 nor CD24 expressing cells were observed. As can be seen from the quadrant plot (FIG. 1B) a good number of cells (98.5%) expressed CD44 indicating a population rich with cells having stemness property that is cancer stem cells.

2. FACS Results for MDA MB231 cells exposed to IC25 drug conc. of Cisplatin for 48 hrs: MDA MB231 cells treated with IC25 drug conc. of Cisplatin were stained with anti-CD44-PE labeled and anti-CD24-FITC labeled antibodies (as seen in FSC-SSC graph FIG. 2A) and the expression was observed in the quadrant plot in the same manner as untreated population. As can be seen from the quadrant plot (FIG. 2B) exposure of Cisplatin IC25 drug conc. did not have much effect on CD44 expressing cell population of MDA MB231 cells indicating that Cisplatin is not very effective on CD44 expressing population that is cancer stem cells.

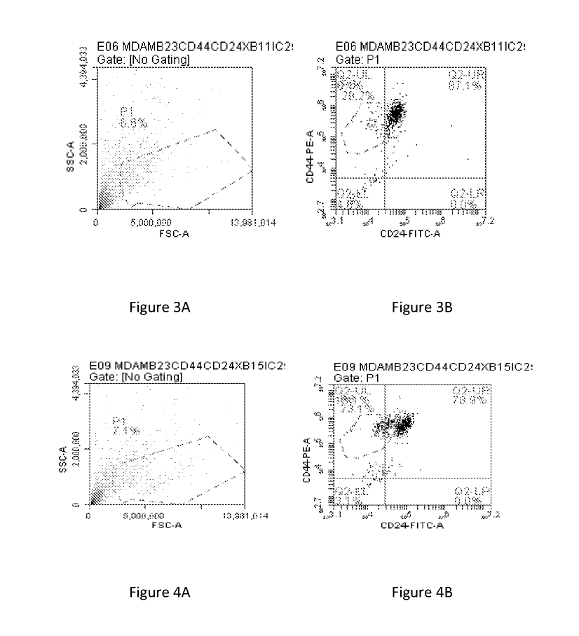

3. FACS Results for MDA MB231 cells exposed to IC25 drug conc. of Ethyl-5-methyl-4-(phenylamino) thieno[2,3-d]pyrimidine-6-carboxylate for 48 hrs: MDA MB231 cells treated with IC25 drug conc. of Ethyl-5-methyl-4-(phenylamino) thieno[2,3-d]pyrimidine-6-carboxylate were stained with anti-CD44-PE labeled and anti-CD24-FITC labeled antibodies (as seen in FSC-SSC graph FIG. 3A) and the expression was observed in the quadrant plot in the same manner as untreated population. As can be seen from the quadrant plot (FIG. 3B) exposure of Ethyl-5-methyl-4-(phenylamino) thieno[2,3-d]pyrimidine-6-carboxylate IC25 drug conc. had a marked effect on the CD44 expressing population of MDA MB231 cells indicating that Ethyl-5-methyl-4-(phenylamino) thieno[2,3-d]pyrimidine-6-carboxylate is very effective on CD44 population that is cancer stem cells population in breast cancer cells.

4. FACS Results for MDA MB231 cells exposed to IC25 drug conc. of Ethyl-5-methyl-4-(4-methylphenyl)amino thieno[2,3-d]pyrimidine-6-carboxylate for 48 hrs: MDA MB231 cells treated with IC25 drug conc. of Ethyl-5-methyl-4-(4-methylphenyl)amino thieno[2,3-d]pyrimidine-6-carboxylate were stained with anti-CD44-PE labeled and anti-CD24-FITC labeled antibodies (as seen in FSC-SSC graph FIG. 4A) and the expression was observed in the quadrant plot in the same manner as untreated population. As can be seen from the quadrant plot (FIG. 4B) exposure of Ethyl-5-methyl-4-(4-methylphenyl)amino thieno[2,3-d]pyrimidine-6-carboxylate) IC25 drug conc. had a marked effect on the CD44 expressing population of MDA MB231 cells indicating that Ethyl-5-methyl-4-(4-methylphenyl)amino thieno[2,3-d]pyrimidine-6-carboxylate) is very effective on CD44 expressing population that is cancer stem cells population in breast cancer cells.

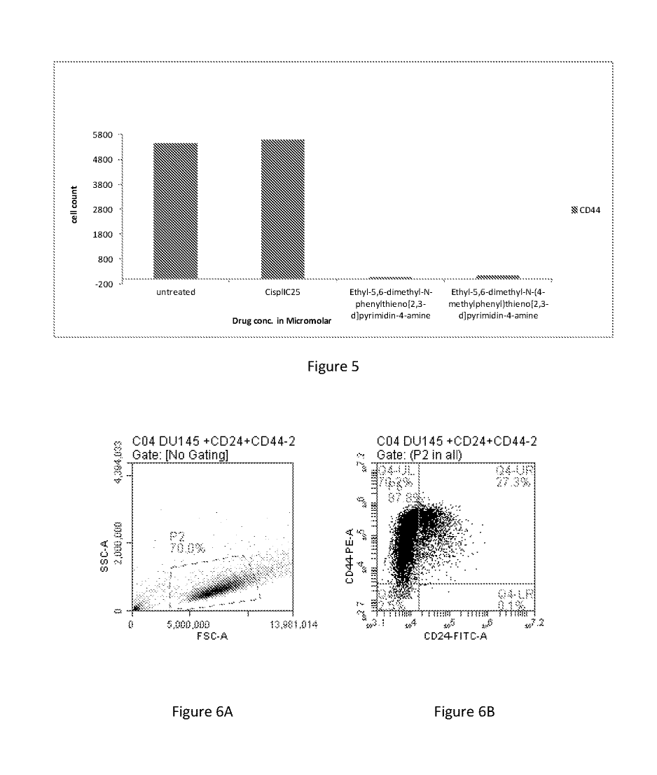

From the above as well as FIG. 5 it is evident that Ethyl-5-methyl-4-(phenylamino) thieno[2,3-d]pyrimidine-6-carboxylate and Ethyl-5-methyl-4-(4-methylphenyl)amino thieno[2,3-d]pyrimidine-6-carboxylate showed much better and enhanced activity on CD44 expressing cells that is cancer stem cells population of MDA MB231 cancer cells compared to standard therapeutic drug Cisplatin. From this it can be inferred that Ethyl-5-methyl-4-(phenylamino) thieno[2,3-d]pyrimidine-6-carboxylate and Ethyl-5-methyl-4-(4-methylphenyl)amino thieno[2,3-d]pyrimidine-6-carboxylate have capability of eradicating or inhibiting proliferation of cancer stem cells in a breast cancer cells.

### Examples 3B

### Study of Effect of Ethyl-5-methyl-4-(phenylamino) thieno[2,3-d]pyrimidine-6-carboxylate; Ethyl-5-methyl-4-(4-methylphenyl)amino thieno[2,3-d]pyrimidine-6-carboxylate and Cisplatin on Prostate Cancer Cell Line DU145 by Flow cytometry Analysis

DU145 is a moderately metastatic prostate cancer cell line. Flow cytometry study was conducted to observe the effect of Ethyl-5-methyl-4-(phenylamino) thieno[2,3-d]pyrimidine-6-carboxylate and Ethyl-5-methyl-4-(4-methylphenyl)amino thieno[2,3-d]pyrimidine-6-carboxylate on DU145 cells compared to standard therapeutic drug Cisplatin.

1. Untreated population: DU145 cells without drug treatment were stained with anti-CD44-PE labeled and anti-CD24-FITC labeled antibodies (as seen in FSC-SSC graph FIG. 6A) and the expression was observed in the quadrant plot. In the quadrant plot upper left region (Q2-UL) only CD44 expressing cells were observed. In the quadrant plot upper right region (Q2-UR) CD44 and CD24 expressing cells were observed. In the quadrant plot lower left region (Q2-LL) only CD24 expressing cells were observed. In the quadrant plot lower right region (Q2-LR) neither CD44 nor CD24 expressing cells were observed. As can be seen from the quadrant plot (FIG. 6B) a good number of cells (87.8%) expressed CD44 indicating a population rich with cells having stemness property that is cancer stem cells.

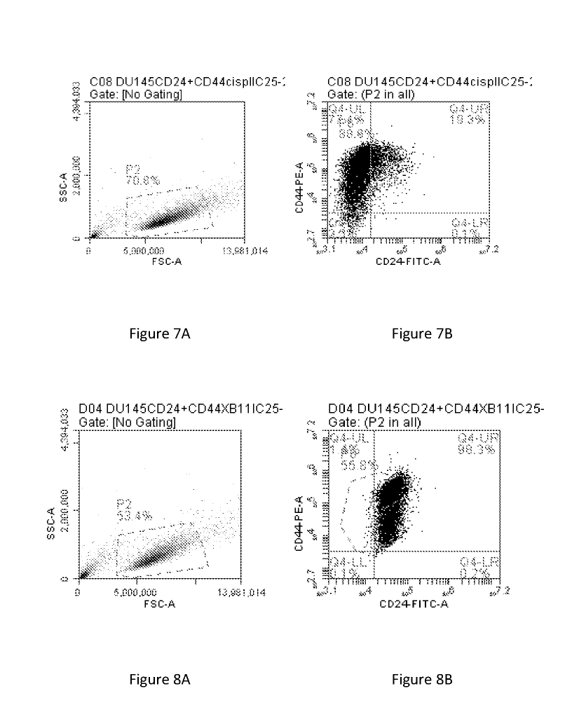

2. FACS Results for DU145 cells exposed to IC25 drug conc. of Cisplatin for 48 hrs: DU145 cells treated with IC25 drug conc. of Cisplatin were stained with anti-CD44-PE labeled and anti-CD24-FITC labeled antibodies (as seen in FSC-SSC graph FIG. 7A) and the expression was observed in the quadrant plot in the same manner as untreated population. As can be seen from the quadrant plot (FIG. 7B) exposure of Cisplatin IC25 drug conc. did not have much effect on CD44 expressing cell population of DU145 cells indicating that Cisplatin is not very effective on CD44 expressing population that is cancer stem cells.

3. FACS Results for DU145 cells exposed to IC25 drug conc. of Ethyl-5-methyl-4-(phenylamino) thieno[2,3-d]pyrimidine-6-carboxylate for 48 hrs: DU145 cells treated with IC25 drug conc. of Ethyl-5-methyl-4-(phenylamino) thieno[2,3-d]pyrimidine-6-carboxylate were stained with anti-CD44-PE labeled and anti-CD24-FITC labeled antibodies (as seen in FSC-SSC graph FIG. 8A) and the expression was observed in the quadrant plot in the same manner as untreated population. As can be seen from the quadrant plot (FIG. 8B) exposure of Ethyl-5-methyl-4-(phenylamino) thieno[2,3-d]pyrimidine-6-carboxylate IC25 drug conc. had a marked effect on the CD44 expressing population of DU145 cells indicating that Ethyl-5-methyl-4-(phenylamino) thieno[2,3-d]pyrimidine-6-carboxylate is very effective on CD44 population that is cancer stem cells population in prostate cancer cells.

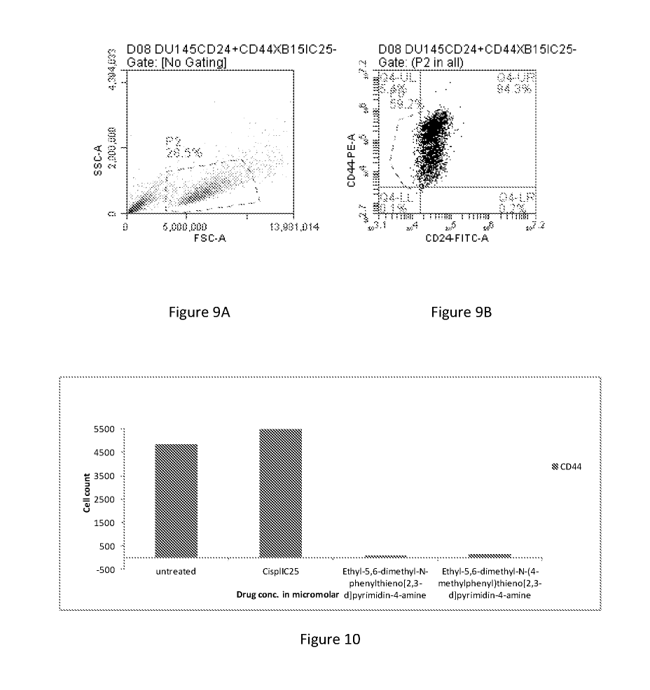

4. FACS Results for DU145 cells exposed to IC25 drug conc. of Ethyl-5-methyl-4-(4-methylphenyl)amino thieno[2,3-d]pyrimidine-6-carboxylate for 48 hrs: DU145 cells treated with IC25 drug conc. of Ethyl-5-methyl-4-(4-methylphenyl)amino thieno[2,3-d]pyrimidine-6-carboxylate were stained with anti-CD44-PE labeled and anti-CD24-FITC labeled antibodies (as seen in FSC-SSC graph FIG. 9A) and the expression was observed in the quadrant plot in the same manner as untreated population. As can be seen from the quadrant plot (FIG. 9B) exposure of Ethyl-5-methyl-4-(4-methylphenyl)amino thieno[2,3-d]pyrimidine-6-carboxylate) IC25 drug conc. had a marked effect on the CD44 expressing population of DU145 cells indicating that Ethyl-5-methyl-4-(4-methylphenyl)amino thieno[2,3-d]pyrimidine-6-carboxylate) is very effective on CD44 expressing population that is cancer stem cells population in prostate cancer cells.

From the above as well as FIG. 10 it is evident that Ethyl-5-methyl-4-(phenylamino) thieno[2,3-d]pyrimidine-6-carboxylate and Ethyl-5-methyl-4-(4-methylphenyl)amino thieno[2,3-d]pyrimidine-6-carboxylate showed much better and enhanced activity on CD44 expressing cells that is cancer stem cells population of DU145 cancer cells compared to standard therapeutic drug Cisplatin. From this it can be inferred that Ethyl-5-methyl-4-(phenylamino) thieno[2,3-d]pyrimidine-6-carboxylate and Ethyl-5-methyl-4-(4-methylphenyl)amino thieno[2,3-d]pyrimidine-6-carboxylate have capability of eradicating or inhibiting proliferation of cancer stem cells in a prostate cancer cells.

1. A compound of formula (I) 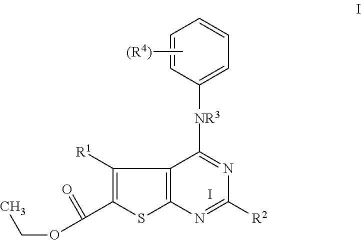
or pharmaceutically acceptable derivative thereof for eradicating or inhibiting proliferation of cancer stem cells, wherein:
each R¹, R² and R³ is independently selected from halogen, C1-6haloalkyl, —CN, —NO₂, —R, —OR, —SR, —N(R)₂, —N(R)NR₂, —C(NR)NR₂, —N(R)C(O)R, C(O)RN(R)₂, —N(R)C(O)N(R)₂, —N(R)C(O)OR, —OC(O)N(R), —N(R)SO₂R, —SO₂RN(R)₂, C(O)R, —C(O)OR, —OC(O)R, —C(O)OR, —S(O)R, or —SO₂R;
each R is independently selected from H, or an optionally substituted group selected from C1-6 aliphatic, a 3-12 membered saturated or partially unsaturated monocyclic carbocyclic ring, phenyl, an 8-12 membered bicyclic aromatic carbocyclic ring; a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic heteroaromatic ring having 1-5 heteroatoms independently selected from nitrogen, oxygen, or sulfur; and
R⁴ is independently selected from —R, —CN, halogen, C1-6 haloalkyl, —NO₂, —SR, —N(R)₂, —N(R)NR₂, —C(NR)NR₂, —N(R)C(O)R, C(O)RN(R)₂, —N(R)C(O)N(R)₂, —N(R)C(O)OR, —OC(O)N(R), —N(R)SO₂R, —SO₂RN(R)₂, C(O)R, —C(O)OR, —C(O)OR, —S(O)R, or —SO₂R;
each R is independently selected from H, or an optionally substituted group selected from C1-6 aliphatic, a 3-12 membered saturated or partially unsaturated monocyclic carbocyclic ring, phenyl, an 8-12 membered bicyclic aromatic carbocyclic ring; a 4-8 membered saturated or partially unsaturated monocyclic heterocyclic ring having 1-2 heteroatoms independently selected from nitrogen, or sulfur, a 5-6 membered monocyclic heteroaromatic ring having 1-4 heteroatoms independently selected from nitrogen or sulfur, or an 8-10 membered bicyclic heteroaromatic ring having 1-5 heteroatoms independently selected from nitrogen or sulfur; and
each n is independently 0-5.

2. The compound as claimed in claim 1, wherein the compound is a pharmaceutically acceptable derivative of compound of formula II or formula III:

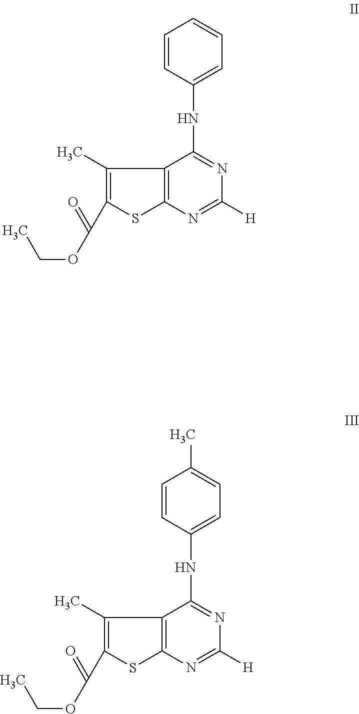 [SMILES: CCOC(=O)c1sc2ncnc(Nc3ccccc3)c2c1C ; CCOC(=O)c1sc2ncnc(Nc3ccc(C)cc3)c2c1C]

3. The compound as claimed in claim 2, wherein the pharmaceutically acceptable derivative of compound of formula II is a compound of formula IV or formula V:

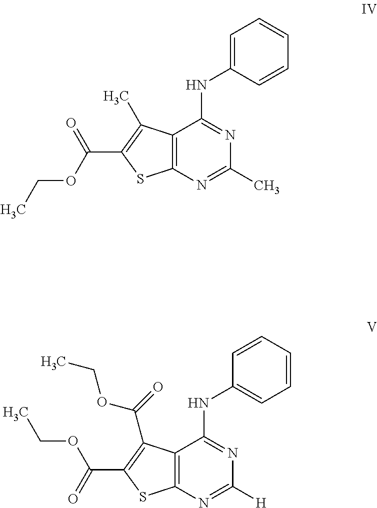 [SMILES: CCOC(=O)c1sc2nc(C)nc(Nc3ccccc3)c2c1C ; CCOC(=O)c1sc2ncnc(Nc3ccccc3)c2c1C(=O)OCC]

4. The compound as claimed in claim 2, wherein the pharmaceutically acceptable derivative of compound of formula III is a compound of formula VI or formula VII:

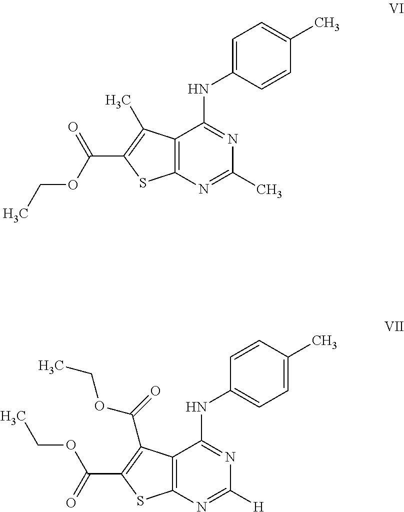

5-14. (canceled)
